(19) United States
(12) Patent Application Publication
(10) Pub. No.: US 2013/0205445 A1
(43) Pub. Date: Aug. 8, 2013

(54) Method of Enhancing the Seed Yield and Promoting the Growth of Plants

(71) Applicants: Ekkehard Neuhaus, Kaiserslautern (DE); Oliver Trentmann, Kaiserslautern (DE); Alexandra Wormit, Aachen (DE); Karina Wingenter, Kaiserslautern (DE)

(72) Inventors: Ekkehard Neuhaus, Kaiserslautern (DE); Oliver Trentmann, Kaiserslautern (DE); Alexandra Wormit, Aachen (DE); Karina Wingenter, Kaiserslautern (DE)

(73) Assignee: Technische Universitat Kaiserslautern, Kaiserslautern (DE)

(21) Appl. No.: 13/637,908

(22) PCT Filed: Dec. 23, 2010
(86) PCT No.: PCT/EP10/07942

§ 371 Date: Dec. 10, 2012

(30) Foreign Application Priority Data
Mar. 27, 2010 (DE) 102010013166.0

(51) Int. Cl. A01H 5/00; C12N 15/11

(52) U.S. Cl. CPC A01H 5/00; C12N 15/11
USPC 800/290; 800/298; 800/306; 800/316; 800/320; 800/320.1; 800/320.2

(57) ABSTRACT

The invention relates to a method of enhancing the seed yield and promoting the growth of monocotyledonous or dicotyledonous plants by overexpression of TMT (tonoplast monosaccharide transporter) protein in osogenic or transgenic plant cells. The invention further concerns a transgenic plant having the property of an enhanced seed yield and increased growth as compared with the wild type, comprising a nucleotide sequence which codes for a TMT (tonoplast monosaccharide transporter) protein, as well as a regulatory nucleotide sequence, operably linked therewith, for the control of an increased gene expression of the TMT protein in the plant cells of the transgenic plant. It further relates to the use of the transgenic plant for the cultivation or production of cultivated plants or useful plants, or biomass, oils or proteins produced therefrom.

### TECHNICAL FIELD

The present invention relates to a method of enhancing the seed yield and promoting the growth of monocotyledonous and dicotyledonous plants by overexpression of a TMT (tonoplast monosaccharide transporter) protein, or a homologue or analogue thereof, in isogenic or transgenic plant cells. The invention relates further to a transgenic plant having the property of an enhanced seed yield and increased growth as compared with the wild type, comprising a nucleotide sequence which codes for a TMT (tonoplast monosaccharide transporter) protein, as well as a. regulatory nucleotide sequence, operably linked therewith, for the control of an increased gene expression of the TMT protein in the plant cells of the transgenic plant. Further, the invention relates to the use of a transgenic plant in the cultivation and production of cultivated plants and useful plants or biomass, oils or proteins produced therefrom. The invention relates further to a gene construct comprising a nucleotide sequence which codes for a TMT (tonoplast monosaccharide transporter) protein TMT1 of Arabidopsis thaliana.

### BACKGROUND ART

Many plants are used as the basis for obtaining oils and proteins and therefore represent important raw material carriers for the foodstuffs industry. In this connection, mention may firstly be made of rape (Brassica napus), which is of worldwide agronomic importance and stores large amounts of oil and protein in its seed. In Germany alone, about 6.2 million tonnes of rapeseed were harvested in 2009. Because of its oil storage qualities, this plant species is particularly desirable in agriculture for the production of oils and proteins, as is demonstrated by many attempts to enhance the seed yield and the yield of the products produced by the plant. Oil production from rapeseed lies in third place worldwide after soybean oil and palm oil (Jaworski et al., 1993, Canola seed yield in relation to harvest methods. In Janick, J E Simon, eds, New Crops. Wiley, New York, p. 330-301). The main ingredients of rapeseed are predominantly oil (about 35-45%) and protein (20-22%). The oil is accordingly the main energy store of the mature grains:

A close relative of rape is the model organism Arabidopsis thaliana, which is used in genetic research and is an excellent representative of the physiology of higher plants. For this reason, the results obtained in the model plant Arabidopsis thaliana can also be transferred to other plant species. Because of the ability of plants to act as oil and protein provider, attempts have already been underway for a relatively long time to enhance the plant yield and the seed yield during cultivation in order ultimately to increase the amount of oil or protein produced during production. If the yields of seed can successfully be enhanced, then an increase in the oil and protein harvest per hectare is accordingly to be expected.

Such an approach is followed in WO 2008/092935 A2. That specification describes a method of enhancing the harvest in plants by modulating the expression of a nucleic acid which codes for the YEP (yield enhancing protein) protein. The YEP is selected from a group of cellular proteins, in particular a nucleosome sampling protein 1-like polypeptide (NAP1-like), a LikeSm polypeptide (Lsm protein), a truncated cyclinH (CycH_{Tr}) polypeptide, a Remorin polypeptide and a DREB protein. Transfection of plant cells with YEP leads to an enhanced growth rate of plants and plant parts such as, for example, seeds. The method is accordingly based on the introduction of foreign genes and the expression thereof in transgenic plants. However, there is no reference to the transport of sugar from the cytosol into the plant vacuole.

In order for developing seeds to be able to store oils and proteins, there must first be communication between the green photosynthetically active leaves and the storage organs; that is to say, the products of photosynthesis are transported from the leaves into the seeds, where they are assembled into lipids and proteins. Photosynthesis is a process in which light energy is converted into biochemical energy and in which sugars, starch, amino acids and organic acids are ultimately produced in the leaf. If the sugars in particular are not removed from the leaf quickly enough, a build-up occurs in the cytosol of the individual photosynthetic cells. This sugar build-up signals to the leaf that the photosynthesis was efficient enough, and it is then down-regulated at genetic level (Rolland et al., 2002, Sugar sensing and signaling in plants, Plant Cell 14: 185-205). This means that the genes necessary for efficient photosynthesis are reduced in their expression by high cytosol sugar concentrations, which leads to a fall in photosynthetic efficiency (Koch, 1996, Carbohydrate-modulated gene expression in plants, Ann Rev Plant Physiol Plant Mol Biol 47: 509-540). This in turn has the result that the maximum achievable photosynthesis is inhibited by high sugar concentrations, here in particular glucose, in the cytosol of the photosynthetically active cells.

In various monocotyledonous and dicotyledonous plants such as Nedicago (identification no. AC131026), Vitis vinifera (identification no. AAX47312) and rice (Oryza sativa; identification no. Os02g13560), a protein has been discovered which is responsible for transporting sugar from the cytoplasm of a plant cell into the vacuole thereof. Plant vacuoles play a central role in the long- or short-term storage of sugars. Storage tissues such as beetroot (Beta vulgaris) and Saccharum officinarum accumulate large amounts of sucrose in order to serve as energy source for plant metabolism. The sugars accumulate in leaves during the day and are released from the vacuole at night.

Finally, a gene has been identified in the plant Arabidopsis whose protein product is a sugar transporter which is localised in the vacuole membrane of photosynthetically active cells and is able to import glucose (monosaccharide) from the cytosol into the vacuole (Wormit et al., 2006, Molecular identification and physiological characterization of a novel monosaccharide transporter from Arabidopsis involved in vacuolar sugar transport. Plant Cell 18: 3476-3490). This transport protein, known as tonoplast monosaccharide transporter (TMT), is localised in the membrane of the largest cell organelle. The vacuole as an organelle of a plant cell occupies a volume of about 90% in a photosynthetically active plant cell (Martinola et al., 2007, Vacuolar transporters and their essential role in plant metabolism, J Exp Bot 58: 83-102) and is therefore of immense importance for the storage of sugar on account of its size alone (Neuhaus H E, 2007, Transport of primary metabolites across the plant vacuolar membrane. FEBS Lett 581: 2223-2226). The tonoplast monosaccharide transporter (TMT) protein comprises three isoforms in Arabidopsis thaliana, which are denoted TMT1, TMT2 and TMT3. The genes for TMT1 and TMT2 exhibit a tissue- and cell-type-specific expression pattern, whereas the TMT3 gene is expressed only very weakly. By means of gene knockouts it has been possible to show that the plants so changed accumulated markedly less glucose and fructose in the vacuole as compared with wild-type plants. No difference could be detected for the disaccharide sucrose.

In the plant species Arabidopsis thaliana alone, more than 60 isoforms of monosaccharide transport proteins have been identified, and these have been classified into various groups (Lalonde et al., 2004, Transport mechanisms for organic forms of carbon and nitrogen between source and sink, Annu, Rev. Plant Biol, 55, 341-372). A number of these transport proteins are likewise localised in the vacuole membrane.

### DISCLOSURE OF INVENTION

Against this background, it is an object of the present invention to provide a method and a transgenic plant with which seed yield and growth can be increased. In this manner, the yield of oil and protein produced with the plant, inter alia, is to be increased.

That object is achieved by a method having the features of claim 1. Preferred embodiments are to be found in the dependent claims.

The inventors have found, surprisingly, that overexpression mutants of the tonoplast monosaccharide transporter (TMT) protein lead to a concentration of sugars such as glucose in the vacuole of plant cells, while the sugar concentrations in the cell cytosol were lower. Overexpression of the sugar transport protein resulted in an enhanced expression of the genes responsible for photosynthesis (also observable by the rise in the photosynthetic efficiency) and ultimately in an increase in the seed weights of the plant. The number of seeds per silique remained unchanged. The number of total seed per plant increased significantly, however. This leads to the conclusion that the overexpression of a sugar transport protein localised in the vacuole membrane results in an increase in the seed yield and in the promotion of growth in plants. Because the transport proteins for transporting mono- and di-saccharides from the cytoplasm into the vacuole are to be found in all vacuole-containing plant cells, the method according to the invention can be used in both monocotyledonous and dicotyledonous plants. If the results obtained in this invention are transferred from Arabidopsis to rape, then the method according to the invention can be used to enhance the oil and protein yield in rape significantly. By means of the invention it is thereby possible to increase the harvests of cultivated and useful plants that are of worldwide importance, and products produced therefrom.

The method according to the invention is based on the overexpression of the tonoplast monosaccharide transporter (TMT) protein, or a homologue or analogue thereof, in isogenic or transgenic cells of monocotyledonous or dicotyledonous plants. The cDNA clone according to the invention (identification no. 8B8T74) codes for 734 amino acids and exhibits a sequence similarity of 32% with the bacterial glucose transporter GTR from Synechocystis and 26% similarity with the Arabidopsis glucose transport protein STP1 localised in the plasma membrane (Wormit et al., 2006, Molecular identification and physiological characterization of a novel monosaccharide transporter from Arabidopsis involved in vacuolar sugar transport. Plant Cell 18: 3476-3490). This protein is known as tonoplast monosaccharide transporter protein (TMT)1. By means of polymerase chain reaction it was possible to amplify two additional TMT isoforms in addition to TMT1, the TMT2 protein containing 739 amino acid residues and the TMT3 protein containing 729 amino acid residues.

The TMT1 protein has 12 transmembrane domains and a relatively large centrally localised hydrophilic loop which connects domains 6 and 7 to one another. This loop contains about 320 amino acid residues and is accordingly four to five times larger than corresponding structures in other known monosaccharide transport proteins in prokaryotes and eukaryotes. The cDNA sequence of the tonoplast monosaccharide transporter (TMT) protein in Arabidopsis thaliana is shown in SEQ ID NO: 2 and the amino acid sequence derived therefrom is shown in SEQ ID NO: 1.

The tests carried out in the following examples clearly show that, by overexpression of the tonoplast monosaccharide transporter (TMT) protein TMT1 of Arabidopsis thaliana, the seed weights could be increased as compared with wild-type plants and the oil and protein contents in mature Arabidopsis seeds increased. Furthermore, the crop yield, of the seeds in various Arabidopsis lines could also be increased. The overexpression further led to Arabidopsis plants developing markedly more quickly as compared with the wild type and exhibiting increased growth. This is presumably attributable to the fact that the overexpression of TMT protein leads to an accumulation of the sugar from the cytosol in the vacuole of the plant cell and thus increases the rate of photosynthesis. Alternatively, it can be assumed that the larger seeds of the overexpression mutants provide the seedlings with more energy, which would represent an advantage over the wild-type seedlings in particular in the early phase of development. Because a number of sugar transport proteins that are localised in the vacuole membrane are known in both monocotyledonous and dicotyledonous plant cells, it is understandable that the principle according to the invention is generally transferable to corresponding sugar transport proteins.

Because the TMT (tonoplast monosaccharide transporter) protein identified in Arabidopsis thaliana also exhibits sequence homologies to transport proteins in other plant cells (see introduction), homologous transport proteins to the TMT1 transport protein of Arabidopsis thaliana are also included in the invention.

A “homologue” within the scope of the invention therefore denotes a transport protein which exhibits a high sequence identity with the TMT (tonoplast monosaccharide transporter) protein TMT1 of Arabidopsis thaliana. Preferably, a homologue has a sequence identity of 50%, 60%, 70%, 75%, 80%, 85%, 90%, 95%, 96%, 97%, 98%, 99% or 100% with an amino acid sequence of Arabidopsis thaliana TMT (tonoplast monosaccharide transporter) protein TMT1.

By way of distinction, an “analogue” within the scope of the invention denotes a sugar transport protein which is localised in the vacuole membrane and performs the function of transporting sugar from the cytoplasm into the vacuole without there necessarily having to be a sequence homology to the TMT (tonoplast monosaccharide transporter) protein. An analogue therefore has the same function as the TMT1 protein but can be structurally different. Unlike a homologue, a genetic relationship is not a prerequisite.

Preferably, the TMT (tonoplast monosaccharide transporter) protein TMT1 of Arabidopsis thaliana having an amino acid sequence according to SEQ ID No 1, or a homologue or analogue thereof, is used to enhance the seed yield to promote the growth of monocotyledonous or dicotyledonous plants. For overexpression or regulated gene expression, a nucleotide sequence which codes for the TMT (tonoplast monosaccharide transporter) protein, or a homologue or analogue thereof, is preferably introduced into the plant cells and overexpressed in the plant cells under the control of a constitutive or inducible promoter. Examples of constitutive promoters are the cauliflower mosaic virus 35S promoter, the Agrobacterium tumefaciens nopaline/octopine synthase promoter or the maize Emu promoter. In addition there are light-inducible promoters, such as, for example, the RUBISCO small subunit rbcS promoter (rice, tomato). The methods for gene expression of genes in plant cells are generally conventionally known and described in the literature, as are the specific cloning methods and the controlled regulation of gene expression. In addition to the promoters, termination sequences for the controlled expression of genes may also be necessary. For the selection of seedlings it is additionally possible to introduce marker genes (e.g. GFP) or other selection genes for which specific antibodies for detection are available, for example against the c-myc motif.

Gene expression can take place either by cloning of a plasmid with the corresponding nucleotide sequences or by incorporation into the genome of the plant cell. Stable integration into the genome of the plant cell and the possibility of targeted regulation of gene expression leads to a stable expression of the TMT (tonoplast monosaccharide transporter) protein, or a homologue or analogue thereof, in the plant cell.

It is possible according to the invention that the overexpression of TMT (tonoplast monosaccharide transporter) protein, or a homologue or analogue thereof, is possible in both isogenic and transgenic plant cells. In isogenic plant cells, the endogenous gene for the transporter protein in question is overexpressed and ultimately leads to enhanced seed production and more rapid growth of the plant as compared with the wild type. In addition, it is also possible to use sugar transport protein from other plant species for the overexpression, so that a transgenic gene expression in the transfected plant cells is obtained. For example, the gene for the TMT (tonoplast monosaccharide transporter) protein TMT1 of Arabidopsis thaliana can be transfected into the rape plant Brassica napus and the gene can be expressed.

The expression of the TMT (tonoplast monosaccharide transporter) protein, or of a homologue or analogue thereof, in plant cells, in particular in plant cells having a high vacuolar storage capacity, such as Brassica napus, is particularly interesting economically because larger amounts of oil or protein can be produced here. For example, endosperm production or potato tuber production can be increased by the targeted insertion of the gene according to the invention for TMT (tonoplast monosaccharide transporter) protein and its expression, as a result of which a higher starch yield is achieved, which in turn increases the production of bioethanol.

The targeted incorporation and the expression of TMT (tonoplast monosaccharide transporter) protein, or a homologue or analogue thereof, in isogenic or transgenic plants cells further leads to an enhancement of the yield per unit area, because the plants reach harvest size much earlier and can accordingly be modified. It is therefore possible to achieve more harvests on the same cultivation area in a shorter time. The method according to the invention leads to marked increases in harvest with a markedly improved biomass yield per unit area as compared with the wild-type plants.

The method according to the invention can in principle be used in all monocotyledonous and dicotyledonous plants. Cultivated plants and useful plants in particular are economically interesting, such as, for example, the rape Brassica napus. Preference is given, for example, to the species and their higher genera of Arabidopsis thaliana, Brassica napus, Brassica oleracea, Brassica rapa, Arachis hypogea, Boechera stricta, Bruguiera gymnorhiza, Citrus paradisi, Poncirus trifoliate, Euphorbia esula, Fragaria vesca, Gossypium hirsutum, Gossypium raimondii, Glycine max, Helianthus annuus, Ipomoea nil, Lycopersicon esculentum, Lactuca perennis, Lactuca saligna, Lactuca serriola, Lactuca sativa, Lotus japonicus, Malus domestica, Medicago truncatula, Nicotiana tabacum, Oryza australiensis, Oryza brachyanth, Oryza punctata, Oryza ridleyi, Oryza rufipogon, Oryza sativa, Populus trichocarpa, Poncirus trifoliata, Prunus persica, Solanum tuberosum Sorghum bicolor, Triticum aestivum, Zea mays, Saccharum officinarum, Panicum virgatum, Miscanthus, Vitis vinifera, Cannabis sativa.

The invention relates further to a transgenic plant having the property of an enhanced seed yield and increased growth as compared with the wild type, comprising a nucleotide sequence which codes for a TMT (tonoplast monosaccharide transporter) protein, as well as a regulatory nucleotide sequence, operably linked therewith, for the control of an increased gene expression of the TMT protein in the plant cells of the transgenic plant. Preferably, the nucleotide sequence codes for TMT (tonoplast monosaccharide transporter) protein TMT1 of Arabidopsis thaliana according to SEQ ID NO: 1, or a homologue or analogue thereof.

The transgenic plant according to the invention can be used in the cultivation of cultivated plants or useful plants. Further, it can be used in the production of biomass, oils or proteins produced therefrom.

The invention also includes the gene construct which comprises a nucleotide sequence which codes for a TMT (tonoplast monosaccharide transporter) protein TMT1 of Arabidopsis thaliana according to SEQ ID NO: 1, or a homologue or analogue thereof, as well as regulatory nucleotide sequences, operably linked therewith, for the control of an increased gene expression of the TMT protein in a plant cell. The invention is explained in greater detail in the following figures.

### BRIEF DESCRIPTION OF DRAWINGS

In the figures

FIG. 1 shows a cloning diagram for the production of an overexpression construct of TMT (tonoplast monosaccharide transporter) protein;

FIG. 2 shows a schematic representation of the T-DNA insertions of the double mutant TMT1-2;

FIG. 3 shows the thousand-kernel weight of Arabidopsis seeds;

FIG. 4 shows oil and lipid contents of Arabidopsis seeds;

FIG. 5 shows the number of seeds per silique in Arabidopsis;

FIG. 6 shows the total weight of the seeds per Arabidopsis plant;

FIG. 7 shows the development of Arabidopsis plants after 15 and 34 days.

### BEST MODE FOR CARYING OUT THE INVENTION

### Examples

Cloning of the Plasmid Construct for the Overexpression of TMT Protein (Attmt1)

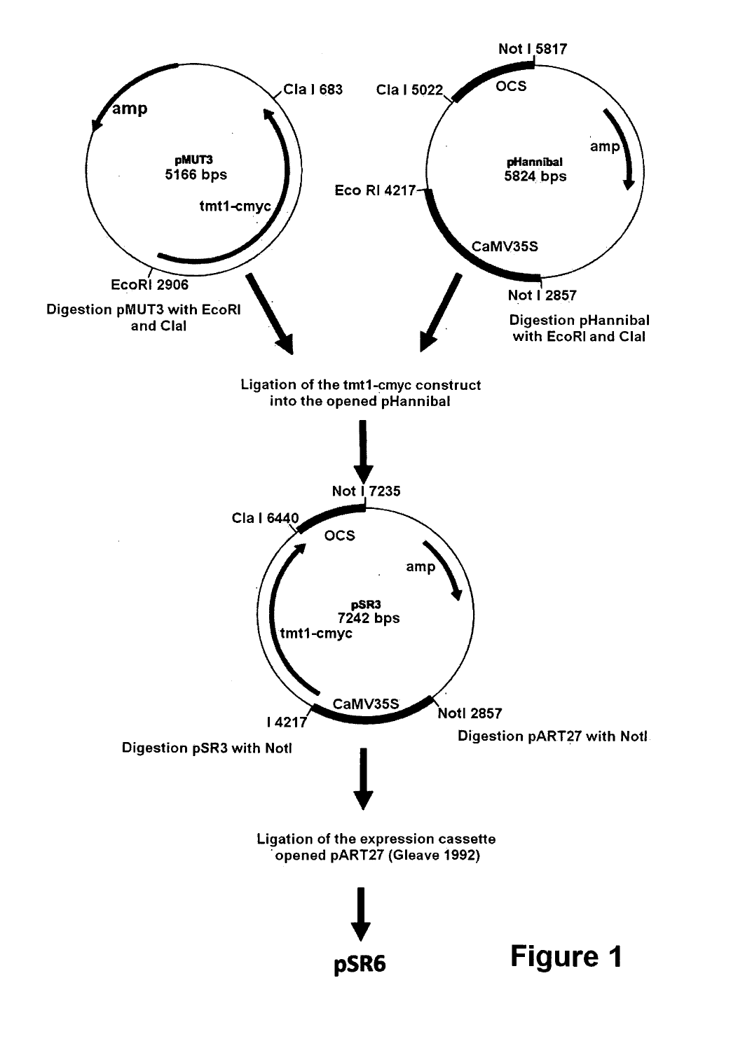

For the expression of the sugar transport protein, the cDNA of TMT (tonoplast monosaccharide transporter) protein TMT1 of Arabidopsis thaliana was used (=Attmt-cDNA). For the production of the overexpression construct, a changed Attmt-cDNA, which contains a c-myc motif in the region of the hydrophilic loop between the putative transmembrane domains 6 and 7, was used. This is the construct pMUT3 (see FIG. 1).

The entire cDNA sequence was first cut from the plasmid pMUT3 with the restriction enzymes EcoRl and Clal and ligated into the plasmid pHannibal linearised with the same enzymes (pSR3). There formed an expression cassette in which a cauliflower mosaic virus 35S promoter was located upstream of the TMT1-cDNA and an OCS terminator was located downstream of the gene at the downstream end (polyadenylation signal of the octopine synthase gene). A further restriction digestion with the restriction enzyme Notl from plasmid pSR3 led to isolation of the cassette, which was finally introduced into the plant transformation vector pART27 (pSR6).

The cDNA sequence of Attmtl (SEQ ID NO: 2) with an integrated c-myc motif (emphasised in red) is as follows:

ATGAAGGGAGCGACTCTCGTTGCTCTCGCCGCCACAATCGGCAATTTC
TTACAAGGATGGGACAATGCCACCATTGCTGGAGCTATGGTTTATATC
AACAAAGACTTGAATCTACCAACCTCTGTTCAAGGTCTTGTCGTTGCT
ATGTCATTGATCGGTGCAACGGTCATCACGACTTGCTCAGGACCGATA
TCTGATTGGCTCGGCAGACGCCCCATGCTCATTTTATCATCAGTTATG
TATTTCGTCTGCGGTTTGATAATGTTGTGGTCTCCCAATGTCTATGTT
CTGTGCTTTGCTAGGCTTCTTAATGGGTTTGGTGCCGGGCTCGCGGTT
ACACTTGTCCCTGTTTACATTTCTGAAACCGCTCCTCCGGAGATCAGA
GGACAGTTAAATACTCTCCCTCAGTTTCTTGGCTCTGGTGGAATGTTT
TTGTCATACTGTATGGTTTTCACTATGTCCCTGAGTGACTCCCCTAGC
TGGAGAGCCATGCTCGGTGTCCTCTCGATCCCTTCTCTTCTTTATTTG
TTTCTCACGGTGTTTTATTTGCCCGAGTCTCCTCGTTGGCTGGTTAGT
AAAGGAAGAATGGACGAGGCTAAGCGAGTTCTTCAACAGTTATGTGGC
AGAGAAGATGTTACCGATGAGATGGCTTTACTAGTTGAAGGACTAGAT
ATAGGAGGAGAAAAAACAATGGAAGATCTCTTAGTAACTTTGGAGGAT
CATGAAGGTGATGATACACTTGAAACCGTTGATGAGGATGGACAAATG
CGGCTTTATGGAACCCACGAGAATCAATCGTACCTTGCTAGACCTGTC
CCAGAACAAAATAGCTCACTTGGGCTACGCTCTCGCCACGGAAGCTTA
GCAAACCAAAGCATGATCCTTAAAGATCCGCTCGTCAATCTTTTTGGC
AGTCTCCACGAGAAGATGCCAGAAGCAGGCGGAAACACTCGGAGTGGG
ATTTTCCCTCATTTCGGAAGCATGTTCAGTACTACTGCCGATGCGCCT
CACGGTAAACCGGCTCATTGGGAAAAGGACATAGAGAGCCATTACAAC
AAAGACAATGATGACTATGCGACTGATGATGGTGCGGAACAAAAACTT
ATCTCCGCAGAAGATTTGCGTAGCCCCTTAATGTCGCGCCAGACCACA
AGCATGGACAAGGATATGATCCCACATCCTACAAGTGGAAGCACTTTA
AGCATGAGACGACACAGTACGCTTATGCAAGGCAACGGCGAAAGTAGC
ATGGGAATTGGTGGTGGTTGGCATATGGGATATAGATACGAAAACGAT
GAATACAAGAGGTATTATCTTAAAGAAGATGGAGCTGAATCTCGCCGT
GGCTCGATCATCTCTATTCCCGGAGGTCCGGATGGTGGAGGCAGCTAC
ATTCACGCTTCTGCCCTTGTAAGCAGATCTGTTCTTGGTCCTAAATCA
GTTCATGGATCCGCCATGGTTCCCCCGGAGAAAATTGCTGCCTCTGGA
CCACTCTGGTCTGCTCTTCTTGAACCTGGTGTTAAGCGTGCCTTGGTT
GTTGGTGTCGGCATTCAAATACTGCAGCAGTTTTCAGGTATCAATGGA
GTTCTCTACTACACTCCTCAGATTCTCGAACGGGCTGGCGTAGATATT
CTTCTTTCGAGCCTCGGACTAAGTTCCATCTCTGCGTCATTCCTCATC
AGCGGYTTAACAACATTACTCATGCTCCCAGCCATTGTCGTTGCCATG
AGACTCATGGATGTATCCGGAAGAAGGTCATTACTTCTCTGGACAATC
CCAGTTCTCATTGTCTCACTTGTCGTCCTTGTCATCAGCGAGCTCATC
CACATCAGCAAAGTCGTGAACGCAGCACTCTCCACAGGTTGTGTCGTG
CTCTACTTCTGCTTCTTCGTGATGGGTTACGGTCCCATTCCAAACATC
CTCTGTTCTGAAATCTTCCCAACAAGAGTCCGTGGTCTCTGCATCGCC
ATATGTGCTATGGTCTTTTGGATTGGAGACATTATTGTCACGTACTCA
CTTCCCGTTCTCCTCAGCTCGATCGGACTAGTTGGTGTTTTCAGCATT
TACGCTGCGGTTTGCGTTATCTCATGGATCTTCGTTTACATGAAAGTC
CCGGAGACTAAAGGCATGCCTTTGGAAGTTATCACAGACTACTTTGCC
TTTGGAGCTCAAGCTCAAGCTTCTGCTCCTTCTAAGGATATATAA

The cloning diagram shown in FIG. 1 shows the production of the overexpression construct pSR6 based on the Attmtl cDNA.

The sequences of the vectors pMUT3 and pSR3 so obtained are given in the sequence listing or SEQ ID NO. 3 and SEQ ID NO. 4.

Production of Attmt1 Overexpression Lines

For the introduction of the overexpression construct into the genome of Arabidopsis plants, the so-called “floral dip process” was used. In this process, inflorescences were dipped in a suspension of agrobacteria (strain GV3101) which had previously been transformed with the construct pSR6. The transformed plants were homozygotic t-DNA insertion cell lines, in which both the native tmtl gene and the native tmt2 gene had been deleted by insertion of a transfer DNA (see Wormit et al., 2006: Molecular identification and physiological characterization of a novel monosaccharide transporter from Arabidopsis involved in vacuolar sugar transport. Plant Cell 18: 3476-3490). In this manner, the production of the native mRNA for tmt1 and tmt2 was suppressed and co-suppression of the artificial mRNA was prevented. Instead of a deletion of the native tmt1 and tmt2 genes, a heterologous tmt sequence can also be overexpressed. The expression of a native tmt1-cDNA sequence is further possible.

When seed formation was complete, the plants were harvested and made to germinate on kanamycin-containing agar plates for the selection of transgenic cell lines. Because the transfected plant cells carry a kanamycin resistance gene, only those seedlings in which transfection with the tmt1-cDNA has been successful can develop, because such cells develop resistance to the antibiotic.

The selection of the overexpression cell lines was carried out by a Northern blot analysis, by selecting those lines which exhibit a marked increase in the Attmtl transcript amount as compared with the wild type.

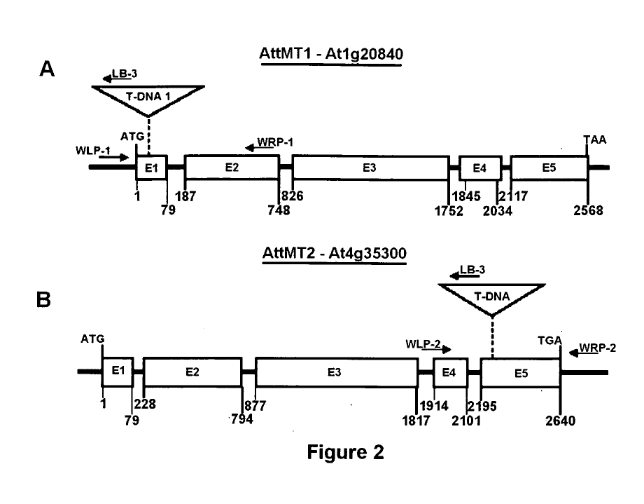

This situation is illustrated again schematically in FIG. 2. The individual t-DNA insertions into the Exon regions of the Attmtl and Attmt2 cDNA are shown.

Cultivation of Plants for Seed Analysis

Before germination, Arabidopsis seeds were incubated for two days in the dark at 4° C. for inhibition. Wild-type plants, the homozygotic mutant cell line tmt1-2 and the three tmt1 overexpression cell lines 1, 4 and 10 were cultivated on plates for nine weeks and cultivated at 22° C. under short-day conditions (10 hours' light, 14 hours' darkness) in a growth chamber. Thereafter, the plants were switched to long-day conditions (14 hours' light, 10 hours' darkness) at 22° C. Watering was continued for three weeks under those conditions. Then watering was stopped and the plants were cultivated further until complete dryness of the infructescence was noted. The seeds of the individual plants were carried out with the aid of a seed collector (Aracon 720).

Seed Analysis

For fatty acid quantification, 0.1 g of fully mature and air-dried seeds was homogenised in a mortar in liquid nitrogen. Then a volume of 1.5 ml of isopropanol was added and further homogenisation was carried out. The suspension was transferred to a reaction vessel having a volume of 1.5 ml and incubated for 12 hours at 4° C. in a laboratory agitator at 100 rpm.

Samples were then centrifuged for 10 min. at 12,000 g and the supernatant was transferred to previously measured 1.5 ml reaction vessels. The reaction vessels were incubated for 8 hours at 60° C. in order to evaporate the isopropanol. The total lipid content was then determined by gravimetry.

For protein quantification of the seed, 0.1 g of seed was homogenised in a mortar at room temperature. Then a volume of 1000 μl of buffer medium (50 mM HEPES, 5 mM MGCl₂, pH 7.5, 1% Triton X100, 15% glycerol, 2% SDS, 1 mM EDTA, PMSF, 1/100 (v/v)) was added and further homogenisation was carried out. The suspension was transferred to 1.5 ml reaction vessels, and the samples were centrifuged for 10 min. at 12,000 g at room temperature. The supernatant was transferred to new 1.5 ml reaction vessels and the proteins were quantified with BCA reagent according to the manufacturer's recommendations.

Results

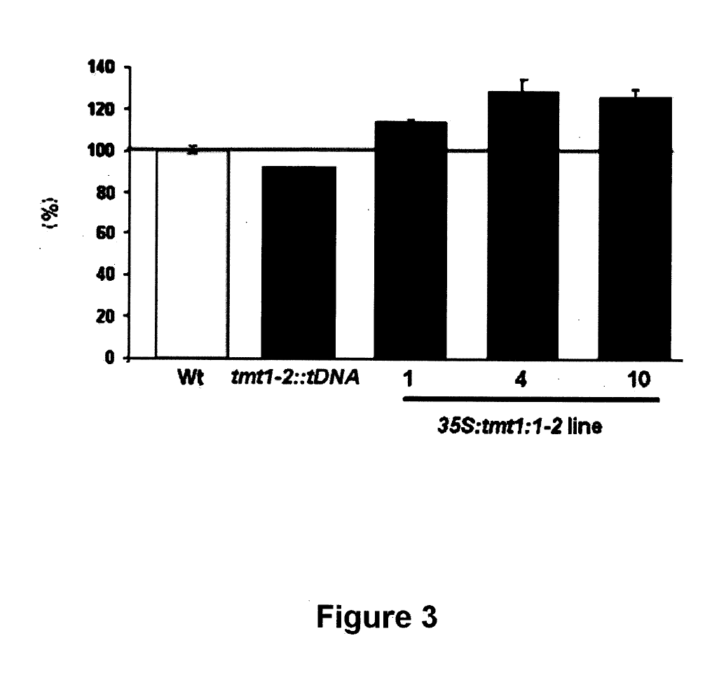

FIG. 3 shows the thousand-kernel weight of Arabidopsis seeds. As control there was used a wild-type Arabidopsis plant as well as a tmt1-2 mutant which exhibited greatly reduced vacuolar TMT activity (Wormit et al., 2006. Molecular identification and physiological characterization of a novel monosaccharide transporter from Arabidopsis involved in vacuolar sugar transport. Plant Cell 18: 3476-3490). Overexpression with overexpression lines 1, 4 and 10 leads to a significant increase in the thousand-kernel weight as compared with the wild type.

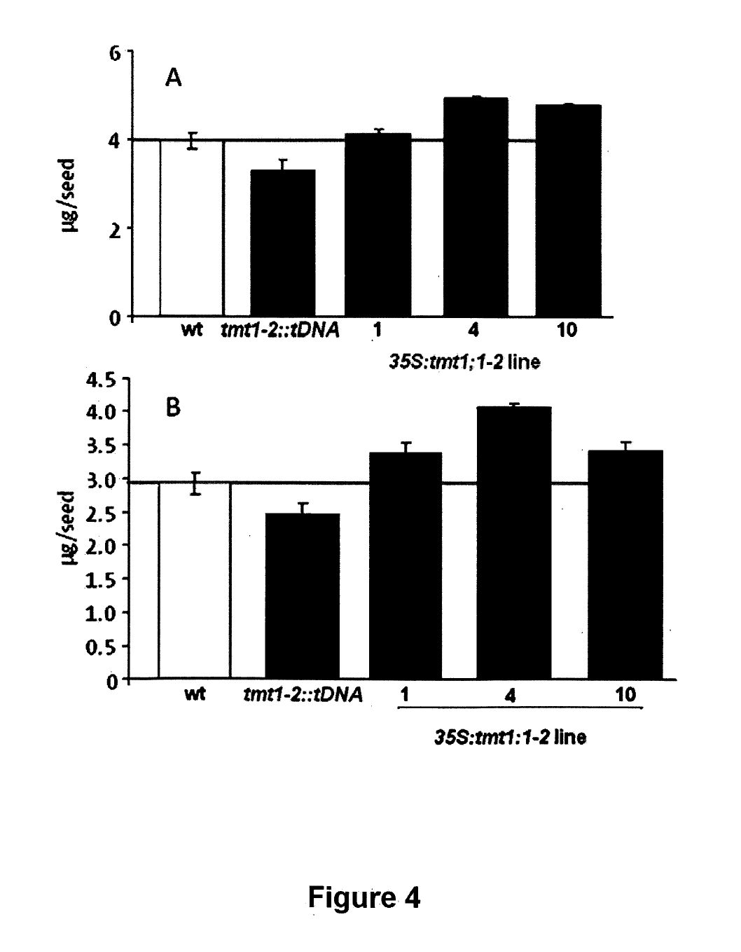

FIG. 4 shows the oil and lipid contents of Arabidopsis seeds. The oil content (FIG. 4A) and the protein content (FIG. 4B) are increased as comₚared with the wild-type Arabidopsis plants (WT) and the three independent TMT1 overexpression lines.

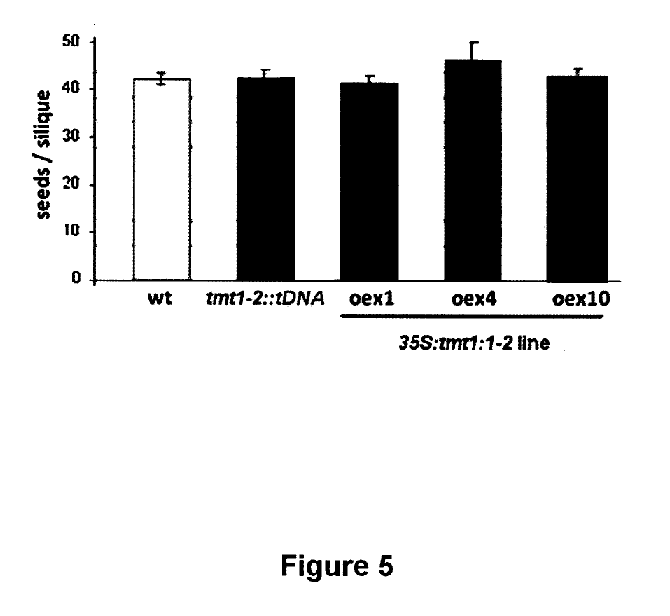

FIG. 5 shows the number of seeds per silique in Arabidopsis. The average number of seeds per silique of wild-type Arabidopsis plants (WT) and the three independent TMT1 overexpression lines is virtually the same.

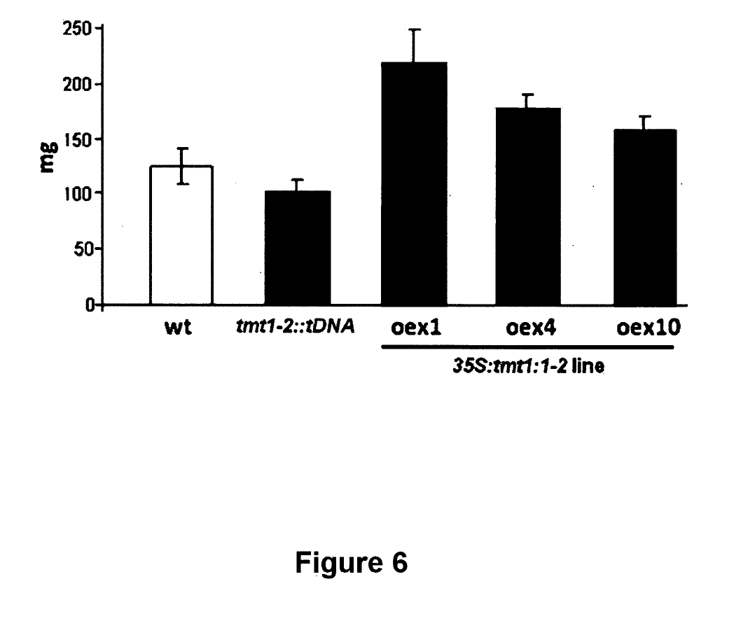

FIG. 6 summarises the total weight of the seeds per Arabidopsis plant. The average total weight of all mature seeds on wild-type Arabidopsis plants (WT) and the three independent TMT1 overexpression lines is shown. The total weight of the seeds is markedly increased in the overexpression lines as compared with the wild type. In FIG. 6A, this can already be seen visually, while in FIG. 6B a quantification of the seeds per plant was carried out.

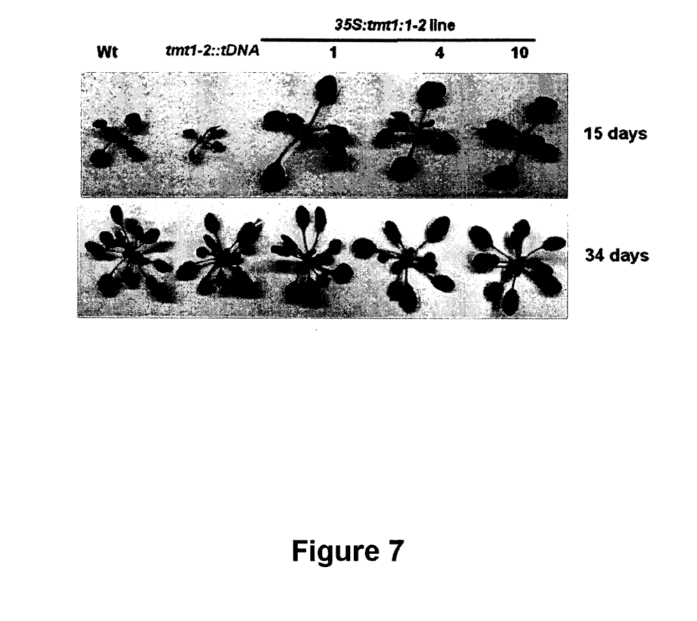

FIG. 7 shows the development of Arabidopsis plants after 15 and 34 days. The overexpression lines exhibit markedly stimulated growth after only 15 days and are markedly larger compared with the wild-type Arabidopsis.plant. An overexpression of TMT1 therefore leads to increased growth of the plants.

In summary, the results clearly show that the seed yield, the germination capacity and the growth of plants can be increased by an overexpression of the TMT (tonoplast monosaccharide transporter) protein.

Text Description Relating to the Figures:

FIG. 1: Cloning diagram for production of the Attmtl overexpression construct pSR6 amp: ampicillin resistance gene; CaMV-35S: cauliflower mosaic virus 35S promoter; OCS: polyadenylation signal from the octopine synthase gene; tmt1-cmyc: cDNA of Attmt1 with inserted cmyc motif;

FIG. 2: Schematic representation of the T-DNA insertions of the double mutant tmt1-2 AttMT1 with insertion of the T-DNA, B: AttMT2 with insertion of the T-DNA;

FIG. 3 Thousand-kernel weight of Arabidopsis seeds. The weight of wild-type Arabidopsis plants (Wt, based on 100%) and three independent TMT1 overexpression lines is shown. tmt1-2 is a mutant which does not exhibit vacuolar TMT activity (Wormit et al., 2006). The data are statistically significant (average values +/− standard error) and are from three independent plant cultivations;

FIG. 4 Oil and lipid contents of Arabidopsis seeds. The oil content (A) and the protein content (B) of wild-type Arabidopsis plants (Wt) and three independent TMT1 overexpression lines are shown. tmt1-2 is a mutant which does not exhibit vacuolar TMT activity (Wormit et al., 2006). The data are statistically significant (average values +/− standard error) and are from three independent plant cultivations;

FIG. 5 Number of seeds per silique in Arabidopsis. The average number of seeds per silique of wild-type Arabidopsis plants (Wt) and three independent TMT1 overexpression lines is shown. tmt1-2 is a mutant which does not exhibit vacuolar TMT activity (Wormit et al., 2006). The data are statistically significant (average values +/− standard error) and are from three independent plant cultivations;

FIG. 6 Total weight of the seeds per Arabidopsis plant. The average total weight of all mature seeds on wild-type Arabidopsis plants (Wt) and three independent TMT1 overexpression lines is shown. tmt1-2 is a mutant which does not exhibit vacuolar TMT activity (Wormit et al., 2006). A. Visual representation, B. Seed quantification. The data are statistically significant (average values +/− standard error) and are from three independent plant cultivations;

FIG. 7 Development of Arabidopsis plants after 15 and 34 days. The development state of wild-type Arabidopsis plants (Wt), tmt1-2 mutants, which lack the activity of the vacuolar glucose transporter TMT (Wormit et al., 2006), and three independent TMT1 overexpression lines is shown. The more rapid development of the TMT overexpression lines can clearly be seen.

1. A method of enhancing the seed yield and promoting the growth of monocotyledonous and dicotyledonous plants by overexpression of TMT (tonoplast monosaccharide transporter) protein, or a homologue thereof, in isogenic or transgenic plant cells, wherein the homologue exhibits a sequence homology with an amino acid sequence of Arabidopsis thaliana TMT (tonoplast monosaccharide transporter) protein TMT1 according to SEQ ID NO:1.

2. The method according to claim 1, characterised in that TMT (tonoplast monosaccharide transporter) protein comprises the amino acid sequence of Arabidopsis thaliana TMT1 according to SEQ ID NO: 1.

3. The method according to claim 1 characterised in that a nucleotide sequence which codes for the TMT (tonoplast monosaccharide transporter) protein, or the homologue thereof is introduced into the plant cells and overexpressed in the plant cells under the control of a constitutive or inducible promoter.

4. The method according to claim 1 characterised in that the TMT (tonoplast monosaccharide transporter)protein exhibits a sequence identity of 50%, 60%, 70%, 75%, 80%, 85%, 90%, 95%, 96%, 97%, 98%, 99% or 100% with an amino acid sequence of Arabidopsis thaliana TMT (tonoplast monosaccharide transporter) protein TMT1 according to SEQ ID NO:1.

5. The method according to claim 1, characterised in that the plants are cultivated or useful plants of the species or higher genera of Arabidopsis thaliana, Brassica napus, Brassica oleracea, Brassica rapa, Arachis hypogea, Boechera stricta, Bruguiera gymnorhiza, Citrus paradisi, Poncirus trifoliate, Euphorbia esula, Fragaria vesca, Gossypium hirsutum, Gossypium raimondii, Glycine max, Helianthus annuus, Ipomoea nil, Lycopersicon esculentum, Lactuca perennis, Lactuca saligna, Lactuca serriola, Lactuca sativa, Lotus japonicus, Malus domestica, Medica go truncatula, Nicotiana tabacum, Oryza australiensis, Oryza brachyanth, Oryza punctata, Oryza ridleyi, Oryza rufipogon, Oryza sativa, Populus trichocarpa, Poncirus trifoliata, Prunus persica, Solanum tuberosum, Sorghum bicolor, Triticum aestivum, ZEA mays, Saccharum officinarum, Panicum virgatum, Miscanthus, Vitis vinifera, Cannabis sativa.

6. A trangenic plant having the property of an enhanced seed yield and increased growth as compared with the wild type, comprising a nucleotide sequence which codes for a TMT (tonoplast monosaccharide transporter) protein, or a homologue thereof, wherein the homologue exhibits a sequence homology with an amino acid sequence of Arabidopsis thaliana TMT (tonoplast monosaccharide transporter) protein TMT1 according to SEQ ID NO: 1, as well as a regulatory nucleotide sequence, operably linked therewith, for the control of an increased gene expression of the TMT protein in the plants cells of the transgenic plant.

7. The transgenic plant according to claim 6 in the cultivation or production of cultivated plants or useful plants, or biomass, oils or proteins produced therefrom.

8. (canceled)

9. (canceled)

10. The transgenic plant according to claim 6, characterised in that the TMT (tonoplast monosaccharide transporter) protein exhibits a sequence identity of 50%, 60%, 70%, 75%, 80%, 85%, 90%, 95%, 96%, 97%, 98%, 99% or 100% with an amino acid sequence of Arabidopsis thaliana TMT (tonoplast monosaccharide transporter) protein TMT1 according to SEQ ID NO: 1.

11. The transgenic plant according to claim 6 characterised in that the plant is selected from Arabidopsis thaliana, Brassica napus, Brassica oleracea, Brassica rapa, Arachis hypogea, Boechera stricta, Bruguiera gymnorhiza, Citrus paradisi, Poncirus trifoliate, Euphorbia esula, Fragaria vesca, Gossypium hirsutum, Gossypium raimondii, Glycine max, Helianthus annuus, Ipomoea nil, Lycopersicon esculentum, Lactuca perennis, Lactuca saligna, Lactuca serriola, Lactuca sativa, Lotus japonicus, Malus domestica, Medicago truncatula, Nicotiana tabacum, Oryza australiensis, Oryza brachyanth, Oryza punctata, Oryza ridleyi, Oryza rufipogon, Oryza sativa, Populus trichocarpa, Poncirus trifoliata, Prunus persica, Solanum tuberosum, Sorghum bicolor, Triticum aestivum, ZEA mays, Saccharum officinarum, Panicum virgatum, Miscanthus, Vitis vinifera, Cannabis sativa.

12. The method according to claim 2 characterised in that a nucleotide sequence which codes for the TMT (tonoplast monosaccharide transporter) protein, or the homologue thereof is introduced into the plant cells and overexpressed in the plant cells under the control of a constitutive or inducible promoter.

13. The method according to claim 2 characterised in that the TMT (tonoplast monosaccharide transporter) protein exhibits a sequence identity of 50%, 60%, 70%, 75%, 80%, 85%, 90%, 95%, 96%, 97%, 98%, 99% or 100% with an amino acid sequence of Arabidopsis thaliana TMT (tonoplast monosaccharide transporter) protein TMT1 according to SEQ ID NO:1.

14. The method according to claim 3 characterised, in that the TMT (tonoplast monosaccharide transporter) protein exhibits a sequence identity of 50%, 60%, 70%, 75%, 80%, 85%, 90%, 95%, 96%, 97%, 98%, 99% or 100% with an amino acid sequence of Arabidopsis thaliana TMT (tonoplast monosaccharide transporter) protein TMT1 according to SEQ ID NO:1.

15. The method according to claim 2 characterised in that the plants are cultivated or useful plants of the species or higher genera of Arabidopsis thaliana, Brassica napus, Brassica oleracea, Brassica rapa, Arachis hypogea, Boechera stricta, Bruguiera gymnorhiza, Citrus paradisi, Poncirus trifoliate, Euphorbia esula, Fragaria vesca, Gossypium hirsutum, Gossypium raimondii, Glycine max, Helianthus annuus, Ipomoea nil, Lycopersicon esculentum, Lactuca perennis, Lactuca saligna, Lactuca serriola, Lactuca sativa, Lotus japonicus, Malus domestica, Medicago truncatula, Nicotiana tabacum, Oryza australiensis, Oryza brachyanth, Oryza punctata, Oryza ridleyi, Oryza rufipogon, Oryza sativa, Populus trichocarpa, Poncirus trifoliata, Prunus persica, Solanum tuberosum, Sorghum bicolor, Triticum aestivum, ZEA mays, Saccharum officinarum, Panicum virgatum, Miscanthus, Vitis vinifera, Cannabis sativa.

16. The method according to claim 3, characterised in that the plants are cultivated or useful plants of the species or higher genera of Arabidopsis thaliana, Brassica napus, Brassica oleracea, Brassica rapa, Arachis hypogea, Boechera stricta, Bruguiera gymnorhiza, Citrus paradisi, Poncirus trifoliate, Euphorbia esula, Fragaria vesca, Gossypium hirsutum, Gossypium raimondii, Glycine max, Helianthus annuus, Ipomoea nil, Lycopersicon esculentum, Lactuca perennis, Lactuca saligna, Lactuca serriola, Lactuca sativa, Lotus japonicus, Malus domestica, Medicago truncatula, Nicotiana tabacum, Oryza australiensis, Oryza brachyanth, Oryza punctata, Oryza ridleyi, Oryza rufipogon, Oryza sativa, Populus trichocarpa, Poncirus trifoliata, Prunus persica, Solanum tuberosum, Sorghum bicolor, Triticum aestivum, ZEA mays, Saccharum officinarum, Panicum virgatum, Miscanthus, Vitis vinifera, Cannabis sativa.

17. The method according to claim 4, characterised in that the plants are cultivated or useful plants of the species or higher genera of Arabidopsis thaliana, Brassica napus, Brassica oleracea, Brassica rapa, Arachis hypogea, Boechera stricta, Bruguiera gymnorhiza, Citrus paradisi, Poncirus trifoliate, Euphorbia esula, Fragaria vesca, Gossypium hirsutum, Gossypium raimondii, Glycine max, Helianthus annuus, Ipomoea nil, Lycopersicon esculentum, Lactuca perennis, Lactuca saligna, Lactuca serriola, Lactuca sativa, Lotus japonicus, Malus domestica, Medicago truncatula, Nicotiana tabacum, Oryza australiensis, Oryza brachyanth, Oryza punctata, Oryza ridleyi, Oryza rufipogon, Oryza sativa, Populus trichocarpa, Poncirus trifoliata, Prunus persica, Solanum tuberosum, Sorghum bicolor, Triticum aestivum, ZEA mays, Saccharum officinarum, Panicum virga tum, Miscanthus, Vitis vinifera, Cannabis sativa.

18. The transgenic plant according to claim 7 characterised in that the TMT (tonoplast monosaccharide transporter) protein exhibits a sequence identity of 50%, 60%, 70%, 75%, 80%, 85%, 90%, 95%, 96%, 97%, 98%, 99% or 100% with an amino acid sequence of Arabidopsis thaliana TMT (tonoplast monosaccharide transporter) protein TMT1 according to SEQ ID NO: 1.

19. The transgenic plant according to claim 7 characterised in that the plant is selected from Arabidopsis thaliana, Brassica napus, Brassica oleracea, Brassica rapa, Arachis hypogea, Boechera stricta, Bruguiera gymnorhiza, Citrus paradisi, Poncirus trifoliate, Euphorbia esula, Fragaria vesca, Gossypium hirsutum, Gossypium raimondii, Glycine max, Helianthus annuus, Ipomoea nil, Lycopersicon esculentum, Lactuca perennis, Lactuca saligna, Lactuca serriola, Lactuca sativa, Lotus japonicus, Malus domestica, Medicago truncatula, Nicotiana tabacum, Oryza australiensis, Oryza brachyanth, Oryza punctata, Oryza ridleyi, Oryza rufipogon, Oryza sativa, Populus trichocarpa, Poncirus trifoliata, Prunus persica, Solanum tuberosum, Sorghum bicolor, Triticum aestivum, ZEA mays, Saccharum officinarum, Panicum virgatum, Miscanthus, Vitis vinifera, Cannabis sativa.

20. The transgenic plant according to claim 10 characterised in that the plant is selected from Arabidopsis thaliana, Brassica napus, Brassica oleracea, Brassica rapa, Arachis hypogea, Boechera stricta, Bruguiera gymnorhiza, Citrus paradisi, Poncirus trifoliate, Euphorbia esula, Fragaria vesca, Gossypium hirsutum, Gossypium raimondii, Glycine max, Helianthus annuus, Ipomoea nil, Lycopersicon esculentum, Lactuca perennis, Lactuca saligna, Lactuca serriola, Lactuca sativa, Lotus japonicus, Malus domestica, Medica go truncatula, Nicotiana tabacum, Oryza australiensis, Oryza brachyanth, Oryza punctata, Oryza ridleyi, Oryza rufipogon, Oryza sativa, Populus trichocarpa, Poncirus trifoliata, Prunus persica, Solanum tuberosum, Sorghum bicolor, Triticum aestivum, ZEA mays, Saccharum officinarum, Panicum virgatum, Miscanthus, Vitis vinifera, Cannabis sativa.


SEQUENCE LISTING (4 sequences)

SEQ ID NO: 1 - protein, 1468 aa, Arabidopsis thaliana
MKGATLVALAATIGNFLQGWDNATIAGAMVYINKDLNLPTSVQGLVVAMSLIGATVITTCSGPISDWLGRRPMLILSSVMYFVCGLIMLWSPNVYVLCFARLLNGFGAGLAVTLVPVYISETAPPEIRGQLNTLPQFLGSGGMFLSYCMVFTMSLSDSPSWRAMLGVLSIPSLLYLFLTVFYLPESPRWLVSKGRMDEAKRVLQQLCGREDVTDEMALLVEGLDIGGEKTMEDLLVTLEDHEGDDTLETVDEDGQMRLYGTHENQSYLARPVPEQNSSLGLRSRHGSLANQSMILKDPLVNLFGSLHEKMPEAGGNTRSGIFPHFGSMFSTTADAPHGKPAHWEKDIESHYNKDNDDYATDDGAGDDDDSDNDLRSPLMSRQTTSMDKDMIPHPTSGSTLSMRRHSTLMQGNGESSMGIGGGWHMGYRYENDEYKRYYLKEDGAESRRGSIISIPGGPDGGGSYIHASALVSRSVLGPKSVHGSAMVPPEKIAASGPLWSALLEPGVKRALVVGVGIQILQQFSGINGVLYYTPQILERAGVDILLSSLGLSSISASFLISGLTTLLMLPAIVVAMRLMDVSGRRSLLLWTIPVLIVSLVVLVISELIHISKVVNAALSTGCVVLYFCFFVMGYGPIPNILCSEIFPTRVRGLCIAICAMVFWIGDIIVTYSLPVLLSSIGLVGVFSIYAAVCVISWIFVYMKVPETKGMPLEVITDYFAFGAQAQASAPSKDIMKGATLVALAATIGNFLQGWDNATIAGAMVYINKDLNLPTSVQGLVVAMSLIGATVITTCSGPISDWLGRRPMLILSSVMYFVCGLIMLWSPNVYVLCFARLLNGFGAGLAVTLVPVYISETAPPEIRGQLNTLPQFLGSGGMFLSYCMVFTMSLSDSPSWRAMLGVLSIPSLLYLFLTVFYLPESPRWLVSKGRMDEAKRVLQQLCGREDVTDEMALLVEGLDIGGEKTMEDLLVTLEDHEGDDTLETVDEDGQMRLYGTHENQSYLARPVPEQNSSLGLRSRHGSLANQSMILKDPLVNLFGSLHEKMPEAGGNTRSGIFPHFGSMFSTTADAPHGKPAHWEKDIESHYNKDNDDYATDDGAGDDDDSDNDLRSPLMSRQTTSMDKDMIPHPTSGSTLSMRRHSTLMQGNGESSMGIGGGWHMGYRYENDEYKRYYLKEDGAESRRGSIISIPGGPDGGGSYIHASALVSRSVLGPKSVHGSAMVPPEKIAASGPLWSALLEPGVKRALVVGVGIQILQQFSGINGVLYYTPQILERAGVDILLSSLGLSSISASFLISGLTTLLMLPAIVVAMRLMDVSGRRSLLLWTIPVLIVSLVVLVISELIHISKVVNAALSTGCVVLYFCFFVMGYGPIPNILCSEIFPTRVRGLCIAICAMVFWIGDIIVTYSLPVLLSSIGLVGVFSIYAAVCVISWIFVYMKVPETKGMPLEVITDYFAFGAQAQASAPSKDI

SEQ ID NO: 2 - DNA, 2205 bp, Arabidopsis thaliana
atgaagggagcgactctcgttgctctcgccgccacaatcggcaatttcttacaaggatgggacaatgccaccattgctggagctatggtttatatcaacaaagacttgaatctaccaacctctgttcaaggtcttgtcgttgctatgtcattgatcggtgcaacggtcatcacgacttgctcaggaccgatatctgattggctcggcagacgccccatgctcattttatcatcagttatgtatttcgtctgcggtttgataatgttgtggtctcccaatgtctatgttctgtgctttgctaggcttcttaatgggtttggtgccgggctcgcggttacacttgtccctgtttacatttctgaaaccgctcctccggagatcagaggacagttaaatactctccctcagtttcttggctctggtggaatgtttttgtcatactgtatggttttcactatgtccctgagtgactcccctagctggagagccatgctcggtgtcctctcgatcccttctcttctttatttgtttctcacggtgttttatttgcccgagtctcctcgttggctggttagtaaaggaagaatggacgaggctaagcgagttcttcaacagttatgtggcagagaagatgttaccgatgagatggctttactagttgaaggactagatataggaggagaaaaaacaatggaagatctcttagtaactttggaggatcatgaaggtgatgatacacttgaaaccgttgatgaggatggacaaatgcggctttatggaacccacgagaatcaatcgtaccttgctagacctgtcccagaacaaaatagctcacttgggctacgctctcgccacggaagcttagcaaaccaaagcatgatccttaaagatccgctcgtcaatctttttggcagtctccacgagaagatgccagaagcaggcggaaacactcggagtgggattttccctcatttcggaagcatgttcagtactactgccgatgcgcctcacggtaaaccggctcattgggaaaaggacatagagagccattacaacaaagacaatgatgactatgcgactgatgatggtgcggaacaaaaacttatctcggcagaagatttgcgtagccccttaatgtcgcgccagaccacaagcatggacaaggatatgatcccacatcctacaagtggaagcactttaagcatgagacgacacagtacgcttatgcaaggcaacggcgaaagtagcatgggaattggtggtggttggcatatgggatatagatacgaaaacgatgaatacaagaggtattatcttaaagaagatggagctgaatctcgccgtggctcgatcatctctattcccggaggtccggatggtggaggcagctacattcacgcttctgcccttgtaagcagatctgttcttggtcctaaatcagttcatggatccgccatggttcccccggagaaaattgctgcctctggaccactctggtctgctcttcttgaacctggtgttaagcgtgccttggttgttggtgtcggcattcaaatactgcagcagttttcaggtatcaatggagttctctactacactcctcagattctcgaacgggctggcgtagatattcttctttcgagcctcggactaagttccatctctgcgtcattcctcatcagcggtttaacaacattactcatgctcccagccattgtcgttgccatgagactcatggatgtatccggaagaaggtcattacttctctggacaatcccagttctcattgtctcacttgtcgtccttgtcatcagcgagctcatccacatcagcaaagtcgtgaacgcagcactctccacaggttgtgtcgtgctctacttctgcttcttcgtgatgggttacggtcccattccaaacatcctctgttctgaaatcttcccaacaagagtccgtggtctctgcatcgccatatgtgctatggtcttttggattggagacattattgtcacgtactcacttcccgttctcctcagctcgatcggactagttggtgttttcagcatttacgctgcggtttgcgttatctcatggatcttcgtttacatgaaagtcccggagactaaaggcatgcctttggaagttatcacagactactttgcctttggagctcaagctcaagcttctgctccttctaaggatatataa

SEQ ID NO: 3 - DNA, 5166 bp, Arabidopsis thaliana
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatttatatatccttagaaggagcagaagcttgagcttgagctccaaaggcaaagtagtctgtgataacttccaaaggcatgcctttagtctccgggactttcatgtaaacgaagatccatgagataacgcaaaccgcagcgtaaatgctgaaaactccaactagttcgatcgagctgaggagaacgggaagtgagtacgtgacaataatgtctccaatccaaaagaccatagcacatatggcgatgcagagaccacggtctgcttgttgggaagatttcagaacagaggatgtttggaatggaccgtaacccatcacgaagaagcagaagtagagcacgacacaacctgtggagagtgctgcgttcacgactttgctgatgtggatgagctcgctgatgacaaggacgacaagtgagacaatgagaactgggattgtccagagaagtaatgaccttcttccggatacatccatgagtctcatggcaacgacaatggctgggagcatgagtaatgttgttaaaccgctgatgaggaatgacgcagagatggaacttagtccgaggctcgaaagaagaatatctacgccagcccgttcgagaatctgaggagtgtagtagagaactccattgatacctgaaaactgctgcagtatttgaatgccgacaccaacaaccaaggcacgcttaacaccaggttcaagaagagcagaccagagtggtccagaggcagcaattttctccgggggaaccatggcggatccatgaactgatttaggaccaagaacagatctgcttacaagggcagaagcgtgaatgtagctgcctccaccatccggacctccgggaatagagatgatcgagccacggcgagattcagctccatcttctttaagataatacctcttgtattcatcgttttcgtatctatatcccatatgccaaccaccaccaattcccatgctactttcgccgttgccttgcataagcgtactgtgtcgtctcatgcttaaagtgcttccacttgtaggatgtgggatcatatccttgtccatgcttgtggtctggcgcgacattaaggggctacgcaaatcttcttccgagataagtttttgttccgcaccatcatcagtcgcatagtcatcattgtctttgttgtaatggctctctatgtccttttcccaatgagccggtttaccgtgaggcgcatcggcagtagtactgaacatgcttccgaaatgagggaaaatcccactccgagtgtttccgcctgcttctggcatcttctcgtggagactgccaaaaagattgacgagcggatctttaaggatcatgctttggtttgctaagcttccgtggcgagagcgtagcccaagtgagctattttgttctgggacaggtctagcaaggtacgattgattctcgtgggttccataaagccgtatttgtccatcctcatcaacggtttcaagtgtatcatcaccttcatgatcctccaaagttactaagagatcttccattgttttttctcctcctatatctagtccttcaactagtaaagccatctcatcggtaacatcttctctgccacataactgttgaagaactcgcttagcctcgtccattcttcctttactaaccagccaacgaggagactcgggcaaataaaacaccgtgagaaacaaataaagaagagaagggatcgagaggacaccgagcatggctctccagctaggggagtcactcagggacatagtgaaaaccatacagtatgacaaaaacattccaccagagccaagaaactgagggagagtatttaactgtcctctgatctccggaggagcggtttcagaaatgtaaacagggacaagtgtaaccgcgagcccggcaccaaacccattaagaagcctagcaaagcacagaacatagacattgggagaccacaacattatcaaaccgcagacgaaatacataactgatgataaaatgagcatggggcgtctgccgagccaatcagatatcggtcctgagcaagtcgtgatgaccgttgcaccgatcaatgacatagcaacgacaagaccttgaacagaggttggtagattcaagtctttgttgatataaaccatagctccagcaatggtggcattgtcccatccttgtaagaaattgccgattgtggcggcgagagcaacgagagtcgctcccttcatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 4 - DNA, 7242 bp, Arabidopsis thaliana
tgaccaagtcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggaaattgtaaacgttaatattttgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggatgccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttggccaagtcggcctctaatacgactcactatagggagctcgtcgagcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgaggaattcgatatgaagggagcgactctcgttgctctcgccgccacaatcggcaatttcttacaaggatgggacaatgccaccattgctggagctatggtttatatcaacaaagacttgaatctaccaacctctgttcaaggtcttgtcgttgctatgtcattgatcggtgcaacggtcatcacgacttgctcaggaccgatatctgattggctcggcagacgccccatgctcattttatcatcagttatgtatttcgtctgcggtttgataatgttgtggtctcccaatgtctatgttctgtgctttgctaggcttcttaatgggtttggtgccgggctcgcggttacacttgtccctgtttacatttctgaaaccgctcctccggagatcagaggacagttaaatactctccctcagtttcttggctctggtggaatgtttttgtcatactgtatggttttcactatgtccctgagtgactcccctagctggagagccatgctcggtgtcctctcgatcccttctcttctttatttgtttctcacggtgttttatttgcccgagtctcctcgttggctggttagtaaaggaagaatggacgaggctaagcgagttcttcaacagttatgtggcagagaagatgttaccgatgagatggctttactagttgaaggactagatataggaggagaaaaaacaatggaagatctcttagtaactttggaggatcatgaaggtgatgatacacttgaaaccgttgatgaggatggacaaatacggctttatggaacccacgagaatcaatcgtaccttgctagacctgtcccagaacaaaatagctcacttgggctacgctctcgccacggaagcttagcaaaccaaagcatgatccttaaagatccgctcgtcaatctttttggcagtctccacgagaagatgccagaagcaggcggaaacactcggagtgggattttccctcatttcggaagcatgttcagtactactgccgatgcgcctcacggtaaaccggctcattgggaaaaggacatagagagccattacaacaaagacaatgatgactatgcgactgatgatggtgcggaacaaaaacttatctcggaagaagatttgcgtagccccttaatgtcgcgccagaccacaagcatggacaaggatatgatcccacatcctacaagtggaagcactttaagcatgagacgacacagtacgcttatgcaaggcaacggcgaaagtagcatgggaattggtggtggttggcatatgggatatagatacgaaaacgatgaatacaagaggtattatcttaaagaagatggagctgaatctcgccgtggctcgatcatctctattcccggaggtccggatggtggaggcagctacattcacgcttctgcccttgtaagcagatctgttcttggtcctaaatcagttcatggatccgccatggttcccccggagaaaattgctgcctctggaccactctggtctgctcttcttgaacctggtgttaagcgtgccttggttgttggtgtcggcattcaaatactgcagcagttttcaggtatcaatggagttctctactacactcctcagattctcgaacgggctggcgtagatattcttctttcgagcctcggactaagttccatctctgcgtcattcctcatcagcggtttaacaacattactcatgctcccagccattgtcgttgccatgagactcatggatgtatccggaagaaggtcattacttctctggacaatcccagttctcattgtctcacttgtcgtccttgtcatcagcgagctcatccacatcagcaaagtcgtgaacgcagcactctccacaggttgtgtcgtgctctacttctgcttcttcgtgatgggttacggtccattccaaacatcctctgttctgaaatcttcccaacaagcagaccgtggtctctgcatcgccatatgtgctatggtcttttggattggagacattattgtcacgtactcacttcccgttctcctcagctcgatcgaactagttggagttttcagcatttacgctgcggtttgcgttatctcatggatcttcgtttacatgaaagtcccggagactaaaggcatgcctttggaagttatcacagactactttgcctttggagctcaagctcaagcttctgctccttctaaggatatataaatcaagcttatcgataagcttggatcctctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaatctgcggccgc